(19) United States
(12) Patent Application Publication
(10) Pub. No.: US 2024/0260902 A1
(43) Pub. Date: Aug. 8, 2024

(54) MODULAR SENSOR PLATFORM FOR INLINE BIOMARKER MONITORING

(71) Applicant: NERv Technology Inc., Kitchener (CA)

(72) Inventors: Khaled Berry, Kitchener (CA); Mazen Zohiry, Kitchener (CA); Ricky Tjandra, Kitchener (CA); Abdallah Hassen El-Falou, Kitchener (CA)

(21) Appl. No.: 18/431,378

(22) Filed: Feb. 2, 2024

Related U.S. Application Data
(60) Provisional application No. 63/482,899, filed on Feb. 2, 2023.

(51) Int. Cl. A61B 5/00; A61M 25/00

(52) U.S. Cl. CPC A61B 5/6852; A61B 5/0002; A61B 5/72; A61M 25/0082; A61B 2560/0443

(57) ABSTRACT

Disclosed herein are devices, processes, and methods for monitoring a fluid for a plurality of signals. Preferably the device comprises one or more sensor modules, a processing module, a flow channel element, and a power source, and is inline with a fluid source, such as a patient catheter. Sensor modules may be swapped in and out of the device and calibrated at a point of care or off-site. The devices, processes, and methods of use, preferably enable continuous patient care at the point of care.

### CROSS-REFERENCE TO RELATED APPLICATIONS

This application claims priority from U.S. Provisional Patent Application No. 63/482,899, filed on Feb. 2, 2023, the disclosure of which is hereby incorporated herein in its entirety by reference.

### FIELD OF THE DISCLOSURE

The present disclosure relates generally to the field of sensors, and more specifically to modular sensors for inline monitoring of biomarkers.

### BACKGROUND

Any discussion of the related art throughout the specification should in no way be considered as an admission that such related art is widely known or forms part of common general knowledge in the field.

WO2021250363 discloses an electronic device for analyzing an analyte present in a fluid, comprising a consumable and interchangeable sensor including temporary receivers capable of interacting with the analyte present in the fluid causing a local property change, a sensor holder in which the sensor is intended to be reversibly positioned and a local property change transducer positioned on the sensor and/or on the sensor holder and suitable for converting the local property change into an electronic signal expressing the local property change. The sensor includes a protection for the temporary receivers. The disclosure also relates to the method for manufacturing this device, as well as to the consumable and interchangeable sensor and the method for manufacturing same. This device is not designed to be compatible with a continuous flow channel, and therefore could not be used for medical analyses at point of care for a patient.

US20200375514 discloses a biosensing device and activation method thereof. The biosensing device includes a sensor module and an electric signal transducer. The sensor module includes a biosensor adapted for measuring a biosignal of a host, and a fixed seat including a conducting member that is electrically connected to the biosensor. The electric signal transducer is for receiving and sending the biosignal measured by the biosensor, is coupled to the sensor module, and includes an electric signal unit electrically connected to the conducting member, and a battery connected to the electric signal unit. The electric signal unit has two electrical contacts that cooperatively define a switch. The battery provides power supply to the biosensor when the electric signal transducer is coupled to the sensor module. Shortcomings of the device include that it is invasive, in that it requires a needle tool to obtain a sample. Furthermore, there is typically only one sensor per device—they are not multiple or swappable.

WO2019183279 discloses sensing systems comprising a replaceable sensor element, a readout system and, optionally, a mount to adhere the device to a patient or to a connected device. Also disclosed herein are methods and design to attach a sensor substrate onto the wearable system conveniently, e.g., magnetically. Also disclosed herein are methods and designs to read and write signals to and from the sensor element while attached to the wearable system for subsequent transmission and processing. Also disclosed herein are designs and methods to create magnetically attachable FPC sensor substrate with a magnetic core. Although the device can measure biofluids present on the skin, it is not adaptable to measure biofluids from a continuous flow channel, and particularly is not adaptable to measuring volumes greater than micro volumes.

KR10-2095356 discloses a biosignal measuring system using a film type multi-channel piezoelectric sensor. The biosignal measuring system comprises: a sensor unit which is provided in an object in contact with a human body and senses a value changing an internal charge characteristic depending on external force to sense biosignals; a signal acquisition unit which converts biosignals into digital signals to generate biosignal data; a main body which accommodates the signal acquisition unit connected to the sensor unit to be able to be detachably attached; and a control unit which stores the biosignal data as a file format and outputs the biosignal data as time series values. Accordingly, the present disclosure provides a multi-channel sensor scalability through a connector separating structure, thereby measuring a plurality of biosignals simultaneously. The system requires that a sensor be in direct contact with the body, which can be invasive. Furthermore, the system is not adaptable to measuring analyte concentrations or provide quantitative results at a specific time. Rather, it relies on measuring changes in the body with time or during a movement.

US20200278313 discloses a sensor assembly and method of using same, wherein a sample is passed through a fluid flow path of a sensor assembly such that the sample intersects at least one sensor comprising at least three electrodes arranged such that two or more electrodes are opposing and two or more electrodes are beside one another. The sensor is read by a reader monitoring changes to the sensor due to the presence of the sample. The reader measures the presence and/or concentration of one or more analytes within the sample based upon data obtained by the reader. Although the device has multiple sensors, they are not swappable or removable. Furthermore, the sample needs to be taken from the patient and then analyzed. This makes the device inadaptable for inline, point of care analysis of a patient's biofluids for a variety of analytes.

U.S. Pat. No. 10,989,341 discloses quick connect fluid connectors with modular connection state sensors removably or detachably mounted thereon. Each modular connection state sensor is configured to sense a connection state of the fluid connector it is mounted on. The connection state sensor indicates that the fluid connector of the first fluid system is connected to the second fluid system prior to initiating fluid flow between the first and second fluid systems. The connection state sensor senses movements of one or more elements of the quick connect fluid connector that is involved in the actual connection of the fluid connector to the second fluid system. The elements that are sensed can be, for example, one or more cylindrical sleeves of the fluid connector or a piston of the fluid connector. Therefore, the connection state of the fluid connectors can be determined accurately. Due to its large scale, the device is not compatible with inline, biofluid analysis or clinical or medical analysis.

U.S. Pat. No. 11,432,747 discloses is an apparatus for measuring bio signals. The apparatus for measuring bio signals includes: a sensor module configured to include a needle sensor to be inserted into the skin; and a main body to which the sensor module is separably coupled, and which is configured to include a controller controlling the sensor module to measure a bio signal through the needle sensor, once the sensor module is coupled. The apparatus is invasive in that it requires a needle tool, and it only has one sensor module per device.

U.S. Pat. No. 8,718,981 discloses a modular sensor assembly in which a sensing module may be packaged and provided separately from a signal processing module and which, in some applications, may facilitate disposal and/or replacement of the sensing module when exposed to a “dirty” or “contaminated” environment without requiring disposal and/or replacement of the entire sensor assembly. In certain applications, the sensing module may include at least one transducer or sensor and a local memory containing a set of conditioning coefficients. The sensing module may be removably coupled to a signal processing module which, in some cases, may be configured to download the set of conditioning coefficients stored in the local memory of the sensing module, and to use the set of conditioning coefficients to produce a substantially linearized output signal. The modular sensor assembly is not a portable device, and therefore may not be adaptable to inline, point of care analysis of a patient's biofluids for a variety of analytes.

In the prior art, there may exist devices that utilize different cartridges, which may contain one or more sensors, paired with a reusable reader. These prior art cartridges are generally disposable and can be manufactured to have different sensors. Cartridges are usually microfluidics based or lateral flow assay (LFA) based, and typically include a calibrator. However, these prior art devices and sensors are not equipped to be fluidically coupled to, or inline with, a patient, and are therefore not capable of making continuous measurements of the patients' biofluids, significantly limiting their applicability in the medical field.

Sensors take up space, which is a premium for a portable device, particularly in clinical applications. Different surgical complications require different sensors, which can't all fit in one device. An ideal solution would be to multiplex all possible sensors onto one sensing platform. This is technologically complex and may not be possible.

Another possible solution is to pick one sensor that can work for most surgical complications, however this may result in lower sensitivity for detecting individual surgical complications.

A best-of-both-worlds solution to this problem may be a device comprising removable and/or hot-swappable sensors. This may preferably allow for custom tailoring of the sensors to the complication, while reducing costs, waste, etc., by reusing the signal processing, communication, and energy source of the system.

All documents cited herein are incorporated by reference.

None of the above cited documents, alone or in combination satisfy the need for a modular sensor assembly that is portable and provides inline, point of care analyses of a person's biofluids, while further addressing the above-mentioned deficiencies.

### BRIEF SUMMARY

It is an object of the disclosure to provide a modular inline device comprising one or more swappable sensor modules, for monitoring a fluid for a plurality of signals.

According to an aspect of the disclosure, there is provided a modular inline device for monitoring a fluid for a plurality of signals, comprising: one or more sensor modules having one or more sensor elements, a processing module, a flow channel element, and a power source; wherein the one or more sensor modules are connectable to the processing module by a releasable connection mechanism; said flow channel element defining fluid communication between a fluid source and one or more sensor elements, the one or more sensor elements configured to measuring signal data related to a fluid property; and said power source providing power to each of the one or more sensor modules and said processing module.

In accordance with an embodiment of the disclosure, the processing module further comprises: a computing module in communication with said sensor module by a communication module, wherein the computing module receives and processes signal data measured in said sensor module.

In accordance with an embodiment of the disclosure, the flow channel element comprises: an inlet port attachable in fluid communication with a catheter, the catheter for insertion in a body of a user, for receiving biofluid from the body of the user and flowing it over the one or more sensor elements; and an outlet port, in fluid communication with said inlet port and with a fluid reservoir, for receiving said biofluid from said inlet port and flowing it into said fluid reservoir.

In accordance with an embodiment of the disclosure, The device the biofluid comprises one or more of: peritoneal fluid, peritoneal drainage fluid, pleural drainage fluid, gastric juice, fecal matter, bile fluid, urine, amniotic fluid, dialysate, sebum, blood, or cerebrospinal fluid.

In accordance with an embodiment of the disclosure, the sensor elements comprise one or more of: pressure, flow and sensors, for measuring pressure, flow and volume of the fluid source in the flow channel element.

In accordance with an embodiment of the disclosure, the sensor modules comprise one or more biosensor elements, the one or more biosensor elements configured to detect signal data relating to one or more biomarkers in the fluid source.

In accordance with an embodiment of the disclosure, the one or more biomarkers comprise one or more of: pH, lactate, electrolytes, impedance, conductivity, dissolved oxygen, dissolved CO₂, temperature, inflammatory markers, enzymes, bacterial proteins, RNA or lipids.

In accordance with an embodiment of the disclosure, the sensor module is physically connected to said processing module by one or more connection mechanisms.

In accordance with an embodiment of the disclosure, the sensor module is wirelessly connected to said processing module by one or more wireless communication mechanisms.

According to an aspect of the disclosure, there is provided a method for monitoring a fluid for a plurality of signals with a modular inline device, the method comprising: releasably connecting, with a connection mechanism, a first sensor module comprising one or more sensor elements, to a processing module; receiving, by an inlet port in fluid communication with a patient, fluid from a fluid source; flowing said fluid through a flow channel element connected to said inlet port and to an outlet port, said flow channel element defining fluid communication between the inlet port and the outlet port; measuring, with said one or more sensor elements, said sensor elements being in fluid communication with said flow channel element, signal data of said fluid source.

In accordance with an embodiment of the disclosure, the method further comprises sending said signal data to a computing module in communication with said sensor module; and processing said signal data with said computing module.

In accordance with an embodiment of the disclosure, the method further comprises: disconnecting the first sensor module; and repeating the method of claim 10 with one or more different sensor modules.

In accordance with an embodiment of the disclosure, the fluid comprises a biofluid, the biofluid originating in a patient, the biofluid comprising one or more of: peritoneal fluid, peritoneal drainage fluid, pleural drainage fluid, gastric juice, fecal matter, bile fluid, urine, amniotic fluid, dialysate, sebum, cerebrospinal fluid, or blood.

In accordance with an embodiment of the disclosure, the sensor modules comprise one or more biosensor elements, the one or more biosensor elements configured to detect signal data relating to one or more biomarkers in the fluid source.

In accordance with an embodiment of the disclosure, the method further comprises: determining, via the processing module, a risk assessment associated with the patient, based on the detection and processing of the one or more biomarkers in the biofluid.

In accordance with an embodiment of the disclosure, the one or more biomarkers comprise one or more of: pH, lactate, electrolytes, impedance, conductivity, dissolved oxygen, dissolved CO₂, temperature, inflammatory markers, enzymes, bacterial proteins, RNA or lipids.

In accordance with an embodiment of the disclosure, the one or more sensor elements comprise one or more of: pressure and flow sensors, for measuring pressure, flow, and volume of the fluid source in the flow channel element.

The advantages and features of the present disclosure will become better understood with reference to the following more detailed description and claims taken in conjunction with the accompanying drawings in which like elements are identified with like symbols.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

In the figures, embodiments are illustrated by way of example. It is to be expressly understood that the description and figures are only for the purpose of illustration and as an aid to understanding.

Embodiments will now be described, by way of example only, with reference to the attached figures, wherein the figures:

FIG. 1 illustrates a modular inline device, in accordance with one embodiment.

FIG. 2 illustrates a sensor module of a modular inline device, in accordance with one embodiment.

FIG. 3 illustrates a processing module in accordance with one embodiment.

FIG. 4 illustrates modular inline device in communication with an exemplary computer system, in accordance with one embodiment.

### DETAILED DESCRIPTION

The term “connected”, “attached”, “affixed” or “coupled to” may include both direct coupling (in which two elements that are coupled to each other contact each other) and indirect coupling (in which at least one additional element is located between the two elements).

Devices and methods for carrying out the disclosure are presented in terms of embodiments depicted within the FIGS. However, the disclosure is not limited to the described embodiments, and a person skilled in the art will appreciate that many other embodiments of the disclosure are possible without deviating from the basic concept of the disclosure, and that any such work around will also fall under scope of this disclosure. It is envisioned that other styles and configurations of the present disclosure can be easily incorporated into the teachings of the present disclosure, and the configurations shall be shown and described for purposes of clarity and disclosure and not by way of limitation of scope.

The features of the disclosure which are believed to be novel are particularly pointed out in the specification. The present disclosure now will be described more fully hereinafter with reference to the accompanying drawings, which are intended to be read in conjunction with both this summary, the detailed description and any preferred and/or particular embodiments specifically discussed or otherwise disclosed. This disclosure may however, be embodied in many different forms and should not be construed as limited to the embodiments set forth herein; rather, these embodiments are provided by way of illustration only and so that this disclosure will be thorough, complete and will fully convey the full scope of the disclosure to those skilled in the art.

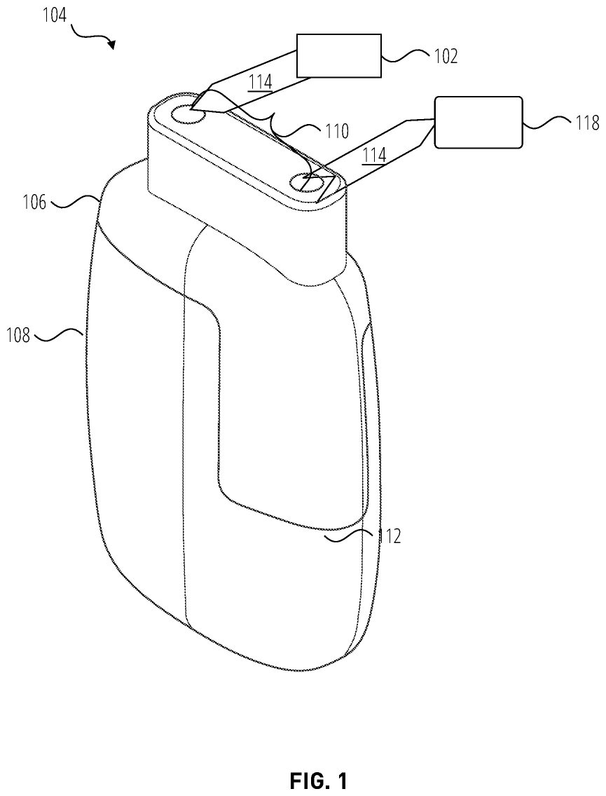

FIG. 1 illustrates modular inline device 104 for monitoring a sample, the device 104 having one or more sensor modules 106, a processing module 108, a flow channel element 110, and a power source.

The modular inline device 104 for monitoring a fluid for a plurality of bioanalyte signals, having one or more sensor modules 106, a processing module 108, and a flow channel element 110, said flow channel element 110 defining fluid communication between a fluid source 102 and said sensor module 106.

Fluid flow direction arrows 114 show fluid flowing into the flow channel element 110 from a fluid source 102 and out of the flow channel element 110, for example into a waste drain.

In the illustrated embodiment, the modular inline device 104 is in an assembled configuration, wherein the sensor module 106 is connected by way of a connection mechanism 112 to the processing module 108.

The fluid source 102 may preferably, but need not necessarily, be a biofluid, which may originate in a patient. Biofluid may comprise, but are not limited to, peritoneal fluid, peritoneal drainage fluid, pleural drainage fluid, gastric juice, fecal matter, bile fluid, urine, amniotic fluid, dialysate, sebum, blood, or cerebrospinal fluid. The fluid source may be connected to said flow channel element 110 element by a medical device, such as a catheter or a drain, which may receive fluid continuously from a patient. Fluid may flow through the modular inline device 104 through the flow channel element 110, and out into a waste reservoir 118 or similar.

The modules 106, 108 preferably fit together by way of a lock-fit connection mechanism 112, which locks them in place.

The locking mechanism could be mechanical (clasps, interference fit, etc.) or magnetic (including electromagnetic mechanisms).

Assembling the modular inline device 104 may comprise locking the sensor module into the processing module. The sensor module may be removed from the processing module, and swapped for a different sensor.

The modular inline device 104 may monitor a patient's biofluids for a variety biomarkers at different times by swapping different sensor modules 106.

The modular inline device 104 is preferably portable, such that it may be easily transported to a patient and used at the point of care.

The modular inline device 104 is preferably inline with a fluid source 102. For example, the modular inline device 104 may be coupled to a patient's catheter for analyzing their biofluid for a variety of biomarkers.

Biofluids may flow continuously through the modular inline device 104 from a drain, catheter, arterial line, urinary catheters, and the like.

A drain may comprise peritoneal drains, pleural drains, cranial drains, and the like.

The connection mechanisms 112 are preferably standard connection mechanisms 112, such as a USB or a simple mechanical connection, in order to allow for different sensors or combinations of sensors to be used with the same processing module 108.

Alternative embodiments may comprise wireless data communication, such as wireless internet or Bluetooth, rather than standard, physical connection mechanisms 112. In this case, the processing module 108 may not need to be physically attached to the sensor module 106.

Additional variations and combinations of the modular inline device 104 may include the sensor module 106 and processing module 108 being in one module, with a separate power module.

Sensor modules 106 can be hot swapped on-site and at the point of care.

Charging of the processing module 108 and calibration of the sensor module 106 may be done separately to enable quicker deployment of the modular inline device 104 at the point of care.

Sensor modules 106 may be equipped with one or more sensors, for measuring and monitoring a plurality of biomarkers and/or bioanalytes in bodily and luminal fluids, which includes, without limitation, peritoneal fluid, peritoneal drainage fluid, pleural drainage fluid, gastric juice, fecal matter, bile fluid, urine, amniotic fluid, dialysate, sebum, or blood. The fluid may be continuously monitored for changes and trends in specific biomarkers, analytes, and biological properties. Examples of these biomarkers, properties and analytes include, but are not limited to, pH, lactate, electrolytes, impedance, conductivity, dissolved oxygen, dissolved CO₂, temperature, inflammatory markers, enzymes, bacterial proteins, RNA or lipids. Systems, methods and devices disclosed herein may be used for various diagnostic applications such as, but not limited to, post-operative leakages, ischemia, infection, and sepsis.

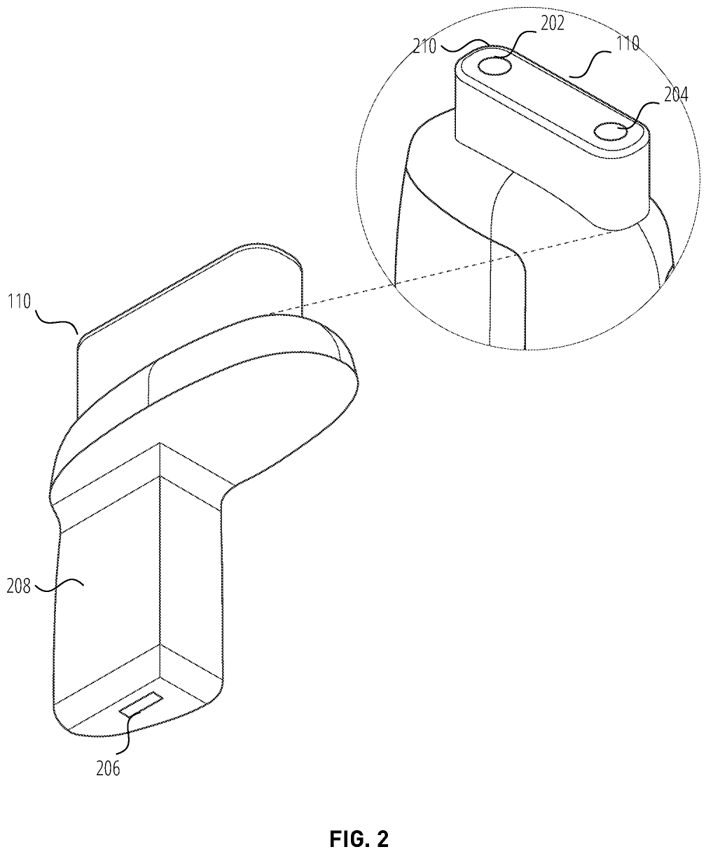

FIG. 2 illustrates the sensor module 106 separated from the processing module 108.

In an embodiment, the flow channel element 110 may be integral to the sensor module 106.

Preferably, the flow channel element 110, defines fluid communication between an inlet port 202 and an outlet port 204.

The inlet port 202 is preferably configured to receive fluid from a fluid source 102, which flows from the inlet port 202 into sensor module 106, and passes over one or more sensor elements 208.

Preferably, but not necessarily, the fluid source 102 is a biofluid source, which is sent to the inlet port 202 from a patient. Patient biofluids preferable flow from the inlet port 202 into sensor module 106, and passes over one or more sensor elements 208.

The sensor elements 208 are configured to sense signals and measure data pertaining to the fluid source 102.

The sensor module 106 may comprise one or more sensor elements 208 for sensing a variety of signals, including, but not limited to, biosignals.

Fluid flows out of the sensor module 106 through an outlet port 204, which is preferably configured to release biofluids to a waste or reservoir.

Preferably, but not necessarily, the one or more sensor elements 208 comprise one or more biosensors, for sensing biomarker signals and measuring bio-signal data from a patient's biofluids.

The sensor module 106 preferably comprises a first connection element 206. This is generally a standard connection mechanism, which allows the sensor module 106 to be attached to the processing module 108.

Generally, the inlet port 202 and outlet port 204 comprise nozzle elements 210, which are sized and configured to fit with a medical device in fluid communication with a patient.

For example, the nozzle elements 210 could connect to a standard catheter or luer lock connections, in order to allow biofluid to flow from a catheter, a drain, or similar, into the inlet port of the modular inline device 104.

There may be provided a variety of different sensor modules 106 for use with a processing module 108, such that a user may swap out the sensor modules 106 and measure a variety of different biomarker signals from a patient's biofluids, without needing to disrupt the patient for each new measurement.

The sensor module may need to be calibrated. It can be calibrated while connected to the processing module 108, at the point of care.

Alternatively, it may be preferable to calibrate the sensor module 106 separately, for example at a calibration station at a lab, or a portable calibration station which may be brought to the point of care.

This is advantageous as it avoids calibration at point of care—a user may bring several, calibrated, and ready-to-use sensor modules 106 with a variety of different sensor elements 208, and may directly analyze a fluid source 102 for one or more signals, analytes, biomarkers, conditions, and the like.

Furthermore, a patient may be monitored continuously, without the need for stopping to calibrate or stopping to change to a new biosensor setup.

The one or more sensor elements 208 preferably include, but are not limited to, pH meters and other potentiometric meters for measuring pH and electrolyte concentration, sensors for conductivity, lactate, amylase, urea, and creatine, and spectrophotometry and spectrometry sensors (including, but not limited to, UV). The sensor module 106 may comprise a light source for making spectrophotometric measurements.

Electrolytes that may be desirable to measure may include sodium, potassium, bicarbonate, and other biologically relevant electrolytes. The sensor elements 208 may also include pressure and flow sensors, for measuring pressure, flow, and volume.

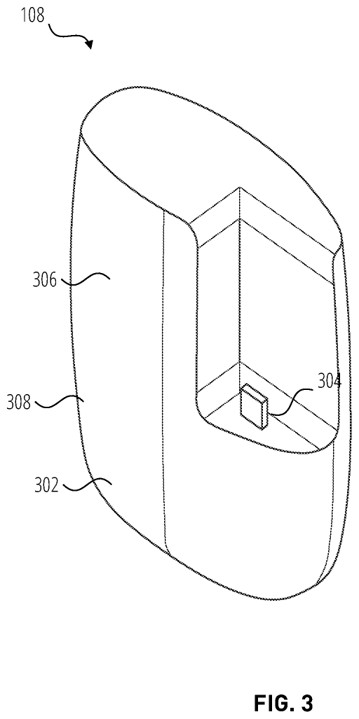

FIG. 3 illustrates the processing module 108.

An advantage of the present disclosure lies in the separability of the sensor module 106 from the processing module 108. Liquid flows through the sensor module 106 and is segregated from the processing module 108, which typically houses electronics and power components that are more readily reusable if they remain dry and uncontaminated by biofluids.

The processing module 108 preferably comprises: a power source 302, a second connection element 304, a computing module 306 in communication with the sensor module 106 by a communication module 308.

The first connection element 206 preferably cooperatively fits with the second connection element 304, creating the connection mechanism 112 which releasably connects the sensor module 106 to the processing module 108. Typically, the second connection element 304 also acts as a power source for the sensor module 106, such that the sensor module 106 does not need to be charged or connected to a battery separately. Alternatively, if the first and second connection elements 206, 304, are simply physical and not electronic mechanisms, a wired connection mechanism 112 may provide power to the sensor module 106 from the processing module 108. Physical connection elements may comprise a friction or interference fit, alignment mechanism, magnets, Velcro™ (hook and loop), clips, adhesives, and any other releasable connection elements known in the art.

Alternatively, in another embodiment, the processing module 108 and sensor module 106 need not be physically interlocked, and could be connected solely by an electronic connection mechanism 112, including, for example, a USB or cable.

The sensor module 106 can communicate with the processing module 108 via a physical connection mechanism 112 or by a wireless communication mechanism 422, including, but not limited to, wireless communication may comprise personal area network (PAN), Bluetooth low energy (BLE), near-field communication (NFC), radio-frequency identification (RFID), wireless internet, wireless broadband communication, and the like. Wired communication may comprise a USB or a cable and the like. The connection mechanism 112, if wired or wireless, may also embody the communication module 308, and may communicate data between the processing module 108 and the computing module 306.

Preferably, the computing module 306 receives and processes data measured in the sensor module 106 by the one or more sensor elements 208. The data may preferably, but not necessarily, be bio-signal data measured in the sensor module 106 by the one or more sensor elements 208, wherein the sensor elements are biosensor elements.

The power source 302 provides power to the processing module 108 and to the sensor module 106 when it is connected to either module. The power source 302 may be rechargeable or disposable, such as a battery. Alternatively, the power source 302 may comprise a cable, such as a plug.

The power source 302 may be provided as a separate module which may be releasably connected to either the processing module 108 or the sensor module 106, or both.

It may alternatively be integral to either the processing module 108, the sensor module 106, or both.

A rechargeable or disposable power source 302 may be charged separately at a charging station, such that power sources 302 connected to processing module 108 or sensor module 106 may be hot-swapped.

This allows for interruption-free and continuous patient monitoring, as new power sources 302 may be brought in as old ones run out of battery, without disconnecting a patient from sensors or flow channels.

The processing module 108 may also be configured to communicate with an external computer, for example by a physical connection, such as a cable, or a wireless connection, for sending the data further processing of data obtained in the sensor module 106 or for charging of the power source 302.

The communication module 308 may comprise a means for wireless communication between the sensor module 106 and the processing module 108 or a means for wired communication between the sensor module 106 and the processing module 108.

The communication module 308 may preferably, but not necessarily, send data obtained from the modular inline device 104 to other devices or people, including, but not limited to, patient emails or applications, health care professional emails or applications, electronic health management systems, cloud databases, artificial intelligence databases, and the like.

The processing module 108 may be equipped with code, or decision making algorithms, with which patient data may be entered, and a risk assessment may be developed. For example, a patient's biofluid pH data may be monitored continuously or at regular intervals following a surgery, and sharp changes in the pH, or specific, indicatory changes at known post-surgical times, may indicate that the patient is at risk of developing an infection. The risk assessment may be made from a combination of sensor data (i.e. pH, blood oxygen, lactate, analyzed together). The processing module 108 may provide a risk assessment, via the communication module 308, to a device.

Wireless communication may comprise personal area network (PAN), Bluetooth low energy (BLE), near-field communication (NFC), radio-frequency identification (RFID), wireless internet, wireless broadband communication, and the like. Wired communication may comprise a USB or a cable and the like.

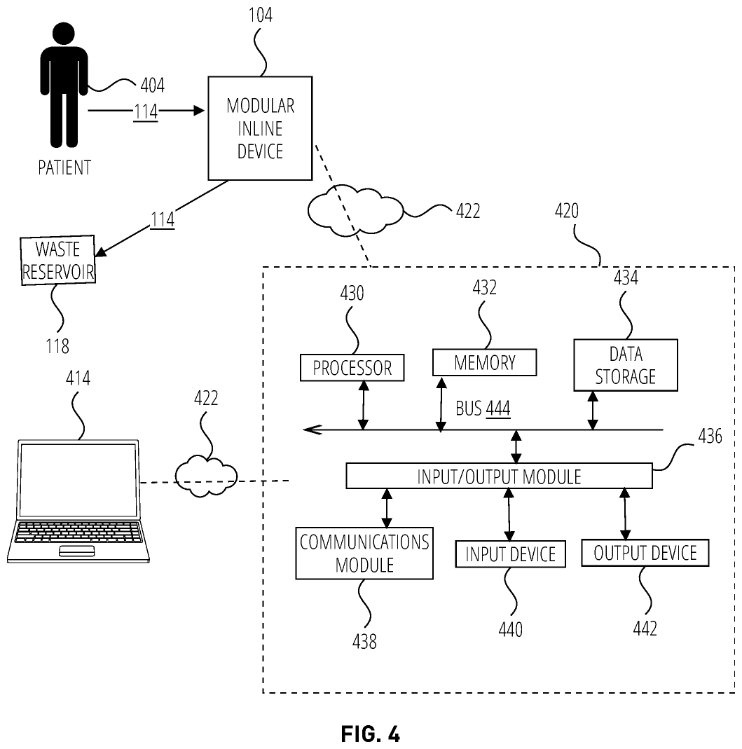

FIG. 4. is a block diagram illustrating an exemplary computer system 420 with which aspects of the disclosure may be implemented. According to an embodiment, fluid flows 114 from a patient 404 to a modular inline device 104, where it flows over sensor elements 208 in the sensor module 106. Data pertaining to bioanalytes in the fluid is measured by the sensor elements 208 and sent to the processing module 108, where the data is processed.

The computer system 420 may be implemented using hardware or a combination of software and hardware, either in a dedicated server, integrated into another entity, or distributed across multiple entities. The computer system 420, or aspects of it, may be integral to the modular inline device 104 and its processing module 108, or separate from it. The processing module 108 may communicate with the computer system 420 via a connection mechanism 112, such as wireless or wired communication mechanisms

Computer system 420 (e.g., server and/or client) includes a bus 444 or other communication mechanism for communicating information, and a processor 430 coupled with bus 444 for processing information. By way of example, the computer system 420 may be implemented with one or more processors 430. Processor 430 may reside in the processing module 108, in the computer system 420, or both. Data may be pre-processed in the processing module 108, for example by correction of data based on on-site conditions, and then sent to a computer system 420 for more rigorous processing. Processor 430 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable entity that can perform calculations or other manipulations of information.

Computer system 420 can include, in addition to hardware, code that creates an execution environment for the computer program in question, e.g., code that constitutes processor firmware, a protocol stack, a database management system, an operating system, or a combination of one or more of them stored in an included memory 432, such as a Random Access Memory (RAM), a flash memory, a Read Only Memory (ROM), a Programmable Read-Only Memory (PROM), an Erasable PROM (EPROM), registers, a hard disk, a removable disk, a CD-ROM, a DVD, or any other suitable storage device, coupled to bus 444 for storing information and instructions to be executed by processor 430. The processor 430 and the memory 432 can be supplemented by, or incorporated in, special purpose logic circuitry. The modular 406 may have its own memory 432 for storing data. The memory 432 may be in a separate computer system 420. The memory 432 may comprise cloud data storage 434.

The instructions may be stored in the memory 432 and implemented in one or more computer program products, i.e., one or more modules of computer program instructions encoded on a computer readable medium for execution by, or to control the operation of, the computer system 420, and according to any method well-known to those of skill in the art, including, but not limited to, computer languages such as data-oriented languages (e.g., SQL, dBase), system languages (e.g., C, Objective-C, C++, Assembly), architectural languages (e.g., Java, .NET), and application languages (e.g., PHP, Ruby, Perl, Python). Instructions may also be implemented in computer languages such as array languages, aspect-oriented languages, assembly languages, authoring languages, command line interface languages, compiled languages, concurrent languages, curly-bracket languages, dataflow languages, data-structured languages, declarative languages, esoteric languages, extension languages, fourth-generation languages, functional languages, interactive mode languages, interpreted languages, iterative languages, list-based languages, little languages, logic-based languages, machine languages, macro languages, metaprogramming languages, multiparadigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, wirth languages, and xml-based languages. Memory 432 may also be used for storing temporary variable or other intermediate information during execution of instructions to be executed by processor 430.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network. The processes and logic flows described in this specification can be performed by one or more programmable processors executing one or more computer programs to perform functions by operating on input data and generating output.

Computer system 420 may further includes a data storage device 434 such as a magnetic disk or optical disk, coupled to bus 444 for storing information and instructions. Computer system 420 may be coupled via input/output module 436 to various devices, including a display element 414, such as a phone screen or computer screen. The input/output module 436 can be any input/output module. Exemplary input/output modules 436 include data ports such a USB ports. The input/output module 436 is configured to connect to a communications module 438. Exemplary communications modules 438 include networking interface cards, such as Ethernet cards and modems. In certain aspects, the input/output module 436 is configured to connect to a plurality of devices, such as an input device 440 and/or an output device 442. Exemplary input devices 440 include a keyboard and a pointing device, e.g., a mouse or a trackball, by which a user can provide input to the computer system 420, Other kinds of input devices 440 can be used to provide for interaction with a user as well, such as a tactile input device, visual input device, audio input device, or brain-computer interface device. For example, feedback provided to the user can be any form of sensory feedback, e.g., visual feedback, auditory feedback, or tactile feedback, and input from the user can be received in any form, including acoustic, speech, tactile, or brain wave input. Exemplary output devices 442 include display devices such as an LCD (liquid crystal display) monitor, for displaying information to the user.

According to one aspect of the present disclosure, the methods for processing analyte data detected by a modular inline device 104, described herein , an be implemented using a computer system 420 in response to processor 430 executing one or more sequences of one or more instructions contained in memory 432. Such instructions may be read into memory 432 from another machine-readable medium, such as data storage device 434. Execution of the sequences of instructions contained in the main memory 432 causes processor 430 to perform the process steps described herein. One or more processors in a multi-processing arrangement may also be employed to execute the sequences of instructions contained in memory 432. In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back end component, e.g., such as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. The communication network can include, for example, any one or more of a LAN, a WAN, the Internet, and the like. Further, the communication network can include, but is not limited to, for example, any one or more of the following network topologies, including a bus network, a star network, a ring network, a mesh network, a star-bus network, tree or hierarchical network, or the like. The communications modules can be, for example, modems or Ethernet cards.

Computer system 420 can include clients and servers. A client and server are generally remote from each other and typically interact through a communication network. The relationship of client and server arises by virtue of computer programs running on the respective computers and having a client-server relationship to each other. Computer system 420 can be, for example, and without limitation, a desktop computer, laptop computer, or tablet computer. Computer system 420 can also be embedded in another device, including the modular inline device 104, for example.

The term “machine-readable storage medium” or “computer readable medium” as used herein refers to any medium or media that participates in providing instructions to processor 430 for execution. Such a medium may take many forms, including, but not limited to, non-volatile media, volatile media, and transmission media. Non-volatile media include, for example, optical or magnetic disks, such as data storage device 434. Volatile media include dynamic memory, such as memory 432. Transmission media include coaxial cables, copper wire, and fiber optics, including the wires that comprise bus 444. Common forms of machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip or cartridge, or any other medium from which a computer can read. The machine-readable storage medium can be a machine-readable storage device, a machine-readable storage substrate, a memory device, a composition of matter effecting a machine-readable propagated signal, or a combination of one or more of them.

As the modular inline device 104 processing module 108 reads and processes sensor data, information may be read from the sensor data and stored in a memory device, such as the memory 432. Additionally, data from the memory 432 may be accessed at a customer server, via a network, such as the bus 444, in order to view bioanalyte concentrations and/or any risk assessments determined by the processing module 108 Further, data storage 434 may be read and loaded into the memory 432. Although data is described as being found in the memory 432, it will be understood that data does not have to be stored in the memory 432 and may be stored in other memory accessible to the processor 430 or distributed among several media, such as the data storage 434, and may be received at a remote or local server for data processing.

In the embodiments shown, the sensor modules 106 are in fluid communication with a patient 404. Upon swapping sensor modules, an old sensor module may be disconnected from the patient, and a new sensor module 106 may be connected to the patient.

Alternatively, it may be preferable to have the processing module 108 in fluid communication with the patient, and the processing module 108 in further fluid communication with the sensor module 106. In this embodiment, when a sensor module 106 is to be replaced with a new sensor module 106, the modular inline device 104 need not be disconnected from the patient. The sensor module 106 may be disconnected from the processing module 108 and a new sensor module 106 may be connected to the processing module 108.

Benefits to having the processing module 108 in fluid communication with the patient may be in the reduced chance of contamination, infection, or spillage if the patient does not need to be disconnected from the device each time a new sensor module 106 is employed.

Alternative embodiments of the modular inline device 104 may include more than two modules. For example, the power source 302 may be a separate module from the sensor module 106 and the processing module 108.

Alternatively, the sensor module 106 and the processing module 108 may be one module, with the power source 302 being a separate module.

It may be desirable to pre-calibrate the sensor modules 106 prior to connecting them to the processing module.

It may be desirable to pre-charge the power source 302 prior to deploying the modular inline device 104.

The sensor modules 106 may be reusable. They may be calibrated and/or cleaned at the point of care, or before and after use, at a secondary location—i.e. in a lab.

In another embodiment, the sensor module 106 may be disposable, although the power source 302 and processing module 108 continue to be reusable, thereby cutting down on unnecessary waste upon disposing of the sensor module 106.

In some embodiments, the modular inline device 104 may comprise a pump, or some similar mechanism, in fluid communication with the flow channel element 110, for pumping fluid through the flow channel element 110, over the sensor elements 208.

In some embodiments, different machine-learning algorithms or techniques may be used, alone or in combination, in the different modules noted above, in order to train the processing module 108 to recognize the various sensor modules 106, to develop calibration procedures optimal to the types of fluid sources 102, and/or to separate multiple analyte signals detected by one sensor element 208, from one another.

These may include, for example, deep learning architectures such as Deep Belief Network (DBN), Stacked Auto Encoder (SAE), Convolutional Neural Network (CNN) or Recurrent Neural Network (RNN) may be used. Other examples include, without limitation, Restricted Boltzmann machines (RBM), Social Restricted Boltzmann Machines (SRBM), Fuzzy Restricted Boltzmann Machines (FRBM), TTRBM models of Deep Belief Networks (DBN) or similar approaches could be used; AE, FAE, GAE, DAE, BAE models of Statistically Adjusted End Use (SAE) models could be used; models such as AlexNet, ResNet, Inception, VGG16, ECNN models of CNN may be used; Bidirectional Recurrent Neural Networks (BiRNN), Long Short-Term Memory (LSTM) networks, Gate Recurrent Unit (GRU) of RNN may also be used.

In some embodiments, other types of algorithms such as physics-based mathematical computations and basic multiple linear regression models may also be relied upon in conjunction with or in complementarity with those architectures and learning algorithms. Many of the functional units described in this specification have been labeled as “engines”, in order to more particularly emphasize their implementation independence. For example, an engine may be implemented as a hardware circuit comprising custom VLSI circuits or gate arrays, off-the-shelf semiconductors such as logic chips, transistors, or other discrete components. An engine may also be implemented in programmable hardware devices such as field programmable gate arrays, programmable array logic, programmable logic devices or the like.

Engines may also be implemented in software for execution by various types of processors. An identified engine of executable code may for instance, comprise one or more physical or logical blocks of computer instructions which may for instance, be organized as an object, procedure, or function. Nevertheless, the executables of an identified engine need not be physically located together, but may comprise disparate instructions stored in different locations which, when joined logically together, comprise the engine and achieve the stated purpose for the module.

Indeed, an engine of executable code may be a single instruction, or many instructions, and may even be distributed over several different code segments, among different programs, and across several memory devices. Similarly, operational data may be identified and illustrated herein within engines, and may be embodied in any suitable form and organized within any suitable type of data structure. The operational data may be collected as a single data set, or may be distributed over different locations including over different storage devices, and may exist, at least partially, merely as electronic signals on a system or network. Where an engine or portions of an engine are implemented in software, the software portions are stored on one or more computer readable storage media.

The present disclosure includes systems having processors to provide various functionality to process information, and to determine results based on inputs. Generally, the processing may be achieved with a combination of hardware and software elements. The hardware aspects may include combinations of operatively coupled hardware components including microprocessors, logical circuitry, communication/networking ports, digital filters, memory, or logical circuitry. The processors may be adapted to perform operations specified by a computer-executable code, which may be stored on a computer readable medium.

The steps of the methods described herein may be achieved via an appropriate programmable processing device or an on-board field programmable gate array (FPGA) or digital signal processor (DSP), that executes software, or stored instructions. In general, physical processors and/or machines employed by embodiments of the present disclosure for any processing or evaluation may include one or more networked or non-networked general purpose computer systems, microprocessors, field programmable gate arrays (FPGA's), digital signal processors (DSP's), micro-controllers, and the like, programmed according to the teachings of the exemplary embodiments discussed above and appreciated by those skilled in the computer and software arts. Appropriate software can be readily prepared by programmers of ordinary skill based on the teachings of the exemplary embodiments, as is appreciated by those skilled in the software arts. In addition, the devices and subsystems of the exemplary embodiments can be implemented by the preparation of application-specific integrated circuits, as is appreciated by those skilled in the electrical arts. Thus, the exemplary embodiments are not limited to any specific combination of hardware circuitry and/or software.

Stored on any one or a combination of computer readable media, the exemplary embodiments of the present disclosure may include software for controlling the devices and subsystems of the exemplary embodiments, for processing data and signals, for enabling the devices and subsystems of the exemplary embodiments to interact with a human user or the like. Such software can include, but is not limited to, device drivers, firmware, operating systems, development tools, applications software, and the like. Such computer-readable media further can include the computer program product of an embodiment of the present disclosure for preforming all or a portion (if processing is distributed) of the processing performed in implementations. Computer code devices of the exemplary embodiments of the present disclosure can include any suitable interpretable or executable code mechanism, including but not limited to scripts, interpretable programs, dynamic link libraries (DLLs), complete executable programs and the like.

Common forms of computer-readable media may include, for example, magnetic disks, flash memory, RAM, a PROM, an EPROM, a FLASH-EPROM, or any other suitable memory chip or medium from which a computer or processor can read.

While particular implementations and applications of the present disclosure have been illustrated and described, it is to be understood that the present disclosure is not limited to the precise construction and compositions disclosed herein and that various modifications, changes, and variations can be apparent from the foregoing descriptions without departing from the spirit and scope of the present disclosure.

As one of ordinary skill in the art will readily appreciate from the disclosure, processes, machines, manufacture, compositions of matter, means, methods, or steps, presently existing or later to be developed, that perform substantially the same function or achieve substantially the same result as the corresponding embodiments described herein may be utilized. Accordingly, the appended claims are intended to include within their scope such processes, machines, manufacture, compositions of matter, means, methods, or steps.

The foregoing descriptions of specific embodiments of the present disclosure have been presented for purposes of illustration and description. They are not intended to be exhaustive or to limit the disclosure and method of use to the precise forms disclosed. Many modifications and variations are possible in light of the above teaching. The embodiments described were chosen and described in order to best explain the principles of the disclosure and its practical application, and to thereby enable others skilled in the art to best utilize the disclosure and various embodiments with various modifications as are suited to the particular use contemplated. It is understood that various omissions or substitutions of equivalents are contemplated as circumstance may suggest or render expedient, but is intended to cover the application or implementation without departing from the spirit or scope of the claims of the present disclosure.

1. A modular inline device for monitoring a fluid for a plurality of signals, comprising: one or more sensor modules having one or more sensor elements, a processing module, a flow channel element, and a power source;
wherein the one or more sensor modules are connectable to the processing module by a releasable connection mechanism;
said flow channel element defining fluid communication between a fluid source and one or more sensor elements, the one or more sensor elements configured to measuring signal data related to a fluid property; and
said power source providing power to each of the one or more sensor modules and said processing module.

2. The device of claim 1, the processing module further comprising:
a computing module in communication with said sensor module by a communication module, wherein the computing module receives and processes signal data measured in said sensor module.

3. The device of claim 2, wherein said flow channel element comprises:
an inlet port attachable in fluid communication with a catheter, the catheter for insertion in a body of a user, for receiving biofluid from the body of the user and flowing it over the one or more sensor elements; and
an outlet port, in fluid communication with said inlet port and with a fluid reservoir, for receiving said biofluid from said inlet port and flowing it into said fluid reservoir.

4. The device of claim 3, wherein the biofluid comprises one or more of: peritoneal fluid, peritoneal drainage fluid, pleural drainage fluid, gastric juice, fecal matter, bile fluid, urine, amniotic fluid, dialysate, sebum, blood, or cerebrospinal fluid.

5. The device of claim 2, wherein the sensor elements comprise one or more of: pressure, flow and sensors, for measuring pressure, flow and volume of the fluid source in the flow channel element.

6. The device of claim 2, wherein said sensor modules comprise one or more biosensor elements, the one or more biosensor elements configured to detect signal data relating to one or more biomarkers in the fluid source.

7. The device of claim 6, wherein the one or more biomarkers comprise one or more of: pH, lactate, electrolytes, impedance, conductivity, dissolved oxygen, dissolved CO₂, temperature, inflammatory markers, enzymes, bacterial proteins, RNA or lipids.

8. The device of claim 1, wherein said sensor module is physically connected to said processing module by one or more connection mechanisms.

9. The device of claim 1, wherein said sensor module is wirelessly connected to said processing module by one or more wireless communication mechanisms.

10. A method for monitoring a fluid for a plurality of signals with a modular inline device, the method comprising:
releasably connecting, with a connection mechanism, a first sensor module comprising one or more sensor elements, to a processing module;
receiving, by an inlet port in fluid communication with a patient, fluid from a fluid source;
flowing said fluid through a flow channel element connected to said inlet port and to an outlet port, said flow channel element defining fluid communication between the inlet port and the outlet port;
measuring, with said one or more sensor elements, said sensor elements being in fluid communication with said flow channel element, signal data of said fluid source.

11. The method of claim 10, further comprising sending said signal data to a computing module in communication with said sensor module;
and
processing said signal data with said computing module.

12. The method of claim 10, further comprising: disconnecting the first sensor module; and
repeating the method of claim 10 with one or more different sensor modules.

13. The method of claim 12, wherein the fluid comprises a biofluid, the biofluid originating in a patient, the biofluid comprising one or more of: peritoneal fluid, peritoneal drainage fluid, pleural drainage fluid, gastric juice, fecal matter, bile fluid, urine, amniotic fluid, dialysate, sebum, cerebrospinal fluid, or blood.

14. The method of claim 12, wherein said sensor modules comprise one or more biosensor elements, the one or more biosensor elements configured to detect signal data relating to one or more biomarkers in the fluid source.

15. The method of claim 14, further comprising:
determining, via the processing module, a risk assessment associated with the patient, based on the detection and processing of the one or more biomarkers in the biofluid.

16. The method of claim 14, wherein the one or more biomarkers comprise one or more of: pH, lactate, electrolytes, impedance, conductivity, dissolved oxygen, dissolved CO₂, temperature, inflammatory markers, enzymes, bacterial proteins, RNA or lipids.

17. The method of claim 10, wherein the one or more sensor elements comprise one or more of: pressure and flow sensors, for measuring pressure, flow, and volume of the fluid source in the flow channel element.
